(19) United States
(12) Patent Application Publication
(10) Pub. No.: US 2016/0367172 A1
(43) Pub. Date: Dec. 22, 2016

(54) BIOLOGICAL INFORMATION MEASURING DEVICE AND METHOD OF CONTROLLING BIOLOGICAL INFORMATION MEASURING DEVICE

(71) Applicant: SEIKO EPSON CORPORATION, Tokyo (JP)

(72) Inventors: Masao KURODA, Shiojiri-shi (JP); Michio NAKADAIRA, Shiojiri-shi (JP); Ichiro AOSHIMA, Matsumoto-shi (JP); Hidenori NAKAMURA, Shimosuwa-machi (JP)

(73) Assignee: SEIKO EPSON CORPORATION, Tokyo (JP)

(21) Appl. No.: 15/117,697

(22) PCT Filed: Feb. 27, 2015
(86) PCT No.: PCT/JP2015/001058

§ 371 Date: Aug. 9, 2016

(30) Foreign Application Priority Data
Mar. 12, 2014 (JP) 2014-048548
Mar. 12, 2014 (JP) 2014-048549

(51) Int. Cl. A61B 5/11; A61B 5/024; A61B 5/00

(52) U.S. Cl. CPC A61B 5/1123; A61B 5/681; A61B 5/4806; A61B 5/024; A61B 2503/10

(57) ABSTRACT

A biological information measuring device includes a biological information detecting unit that detects biological information of a user; a motion information detecting unit that detects motion information related to the motion of the user; an informing unit that informs the user of a load state of the user; a condition setting unit that sets informing condition of the load state for the kind of behavior; a behavior analysis unit that analyzes the kind of behavior of the user based on the detected biological information and motion information; a condition determining unit that determines whether the informing condition is satisfied according to the kind of detected behavior of the user which is analyzed based on the detected biological information and motion information; and an informing control unit that informs the informing unit of the load state when it is determined that the informing condition is satisfied.

### CROSS-REFERENCES TO RELATED APPLICATIONS

This application claims priority to Japanese Patents Application No. 2014-048548, filed Mar. 12, 2014, and No. 2014-048549, filed Mar. 12, 2014, all of which are herein incorporated by reference.

### BACKGROUND

### 1. Technical Field

The present invention relates to a biological information measuring device and a method of controlling the biological information measuring device.

### 2. Related Art

In the related art, a biological information processing device that is formed of a pulsimeter, sets a target zone (range of pulse rate) of an exercise for each person in an initial calibration process, determines whether a calculated pulse rate acquired by the pulsimeter is a pulse rate within the set target zone, controls lighting of a light emitting diode (LED) lamp in a blue color when the pulse rate is within the target zone, and controls lighting of the LED lamp in a red color when the pulse rate is out of the target zone is known (for example, JP-A-2013-22256).

In the biological information processing device disclosed in JP-A-2013-22256, exercise intensity (load state) is informed by changing the color of one lamp lighting according to the zone described above and the current pulse rate. In regard to such a biological information processing device used by being worn on a human body (for example, a wrist), it is suggested that the load state thereof is informed using vibration of the device main body.

However, the pulse rate is increased in a case where a person has a psychological load (a case where a person has a stress under a state of extreme tension or the like) as well as in a case where a person does an exercise.

Accordingly, in the configuration of JP-A-2013-22256, a body motion is detected in the same manner as the pulses rate based on a pulse wave and the load state is not informed when a user does not do an exercise. For this reason, a biological information measuring device capable of informing of the load state even when the user does not do an exercise has been demanded.

However, in the case where the user is not in a state of exercising, when the load state is informed if the condition applied to the case where the user is in a state of exercising is satisfied, there is a concern that the load state is informed at a timing inappropriate for the user's actual behavior. In such a case, there is a problem in that the load state of the user may not be effectively informed.

Further, although the kind of exercise varies for each user, when the load state of the user is informed in a case where the body motion exceeds the upper limit of the range of one piece of predetermined biological information or the body motion is lower than the lower limit thereof as the biological information processing device disclosed in JP-A-2013-22256, there is a problem in that the load state according to the kind of exercise of the user may not be effectively informed.

### SUMMARY

An advantage of some aspects of the invention is to provide a biological information measuring device capable of effectively informing the load state of a user and a method of controlling the biological information measuring device.

A first aspect of the invention is directed to a biological information measuring device including: a biological information detecting unit that detects biological information of a user; a motion information detecting unit that detects motion information related to the motion of the user; an informing unit that informs the user of a load state of the user; a behavior analysis unit that analyzes the kind of behavior of the user based on the detected biological information and motion information; a condition setting unit that sets informing condition of the load state for the kind of behavior; a condition determining unit that determines whether the informing condition is satisfied according to the kind of analyzed behavior of the user based on the detected biological information and motion information; and an informing control unit that informs the informing unit of the load state when it is determined that the informing condition is satisfied by the condition determining unit.

Exercise and non-exercise can be exemplified as the kind of behavior; a pulse wave, a body temperature, blood pressure, an electrocardiogram, and a brain wave can be exemplified as biological information; and an acceleration value acting on the biological information measuring device according to motion of the user can be exemplified as the motion information. Further, the pulse and heartbeat measured based on the pulse wave are strictly different from each other, but the pulse rate may be replaced with the heartbeat because the pulse and the heartbeat have substantially the same meaning for a healthy person.

According to the first aspect, determination of whether to inform the user of the load state of the user is made based on the informing condition according to the kind of behavior of the user which is analyzed based on the detected biological information and the motion information, and then the load state is informed by the informing unit in the case where the condition is satisfied. According to this, since the informing condition is set according to the kind of current behavior of the user and the load state is informed in the case where it is determined that the informing condition is satisfied, informing according to the kind of current behavior of the user can be performed. Accordingly, the load state of the user can be effectively informed and the convenience of the biological information measuring device can be increased.

In the first aspect, it is preferable that the condition setting unit sets the detected biological information to exceed a lower limit of a range of the biological information to be set according to the user as the informing condition in a case where the kind of behavior is at least one of exercise and non-exercise.

According to the first aspect with this configuration, in the case where the kind of behavior is at least one of exercise and non-exercise, the load state of the user is informed when the detected biological information exceeds the lower limit of the range of the biological information to be set according to the user.

In this manner, in the case where the kind of behavior of the user is exercise and the range of the biological information to be set according to the user is a range in which fat burning efficiency is increased, it is possible to encourage the user to maintain the behavior when the load state is informed. Therefore, it is possible to make the user maintain the state with high fat burning efficiency.

Meanwhile, in the case where the kind of behavior of the user is non-exercise and the range of the biological information to be set according to the user is a range in which the user has a psychological load, the user can recognize that he or she has a psychological load, that is, he or she is under stress when the load state is informed, so that the user can objectively recognize the factor causing the stress. Moreover, since there is a possibility that concentration of the user can be improved and greater ability than usual can be exhibited when the user is under a moderate degree of stress, the user can try to maintain the moderate degree of stress.

In addition, in the case where the kind of behavior is non-exercise, the upper limit and the lower limit of the range of the biological information to be set as the informing condition may be the same as each other. In this case, since the load state is informed at the time of exceeding the threshold value, the user can recognize that he or she has a psychological load.

Therefore, the load state can be effectively informed in both cases where the kind of behavior of the user is exercise and the kind of behavior of the user is non-exercise.

In the first aspect, it is preferable that the condition setting unit sets the detected biological information to exceed an upper limit of the range of the biological information to be set according to the user as the informing condition in the case where the kind of behavior is at least one of exercise and non-exercise.

According to the first aspect with this configuration, in the case where the kind of behavior is at least one of exercise and non-exercise, when the detected biological information exceeds the upper limit of the range of the biological information to be set according to the user, the load state of the user is informed. In this manner, in a case where the kind of behavior of the user is exercise and the range of the biological information is a range in which the fat burning efficiency is increased, it is possible to encourage the user to correct the behavior when the biological information is informed. That is, it is possible to make the user recognize that the current load state of the user is not appropriate and the fat burning efficiency of the exercise is not high.

Meanwhile, in the case where the kind of behavior of the user is non-exercise and the range of the biological information to be set according to the user is the range in which the user has a psychological load, the user can recognize that he or she has a psychological load which exceeds the upper limit, that is, he or she is under extreme stress when the load state is informed. Accordingly, it is possible to encourage the user to eliminate the factor of the stress.

Therefore, the load state can be effectively informed in both cases where the kind of behavior of the user is exercise and the kind of behavior of the user is non-exercise.

In the first aspect, it is preferable that regulation is made such that the condition setting unit sets the detected biological information to be lower than the lower limit of the range of the biological information to be set according to the user as the informing condition in the case where the kind of behavior is exercise and the detected biological information to be lower than the lower limit of the range of the biological information to be set according to the user as the informing condition in the case where the kind of behavior is non-exercise.

According to the first aspect with this configuration, in the case where the kind of behavior is exercise, when the detected biological information is lower than the lower limit of the range of the biological information, the load state of the user is informed. In this manner, in a case where the range of the biological information is a range in which the fat burning efficiency is increased, it is possible to encourage the user to correct the behavior when the biological information being lower than the range is informed. That is, it is possible to make the user recognize that the current load state of the user is not appropriate and the fat burning efficiency of the exercise is low.

Meanwhile, in the case where the kind of behavior of the user is non-exercise, informing the user of the load state is regulated when the detected biological information is lower than the lower limit of the range of the biological information. In this manner, the psychological load of the user is alleviated, and the load state is not informed when the user is calm. Accordingly, it is possible to prevent the load state from being increased by the user being excited again or the like and to maintain a stress-free state.

In the first aspect, it is preferable that the condition setting unit includes the upper limit of the range of the biological information which is lower than that of the case where the kind of behavior is exercise in the informing condition in the case where the kind of behavior is non-exercise.

Here, in the case where the kind of behavior is non-exercise, the change of the biological information, particularly, the upper limit of the biological information tends to be low compared to the case where the kind of behavior is exercise. For example, there is such a tendency when the biological information is the pulse rate based on the pulse wave.

For this reason, according to the first aspect, in the case where the kind of behavior is non-exercise, the upper limit of the range of the biological information to be set according to the user is set to be lower than the upper limit of the range of the biological information to be set when the kind of behavior is exercise. In this manner, the change of the biological information at the time of non-exercise, whose width of the change of the biological information is smaller than that at the time of exercise, can be reliably detected. Therefore, the load state of the user can be accurately informed.

In the first aspect, it is preferable that the biological information measuring device further includes an informing regulation unit that regulates informing using the informing unit in a case where the kind of analyzed behavior of the user is sleep.

According to the first aspect with this configuration, in the case where the kind of behavior of the user is sleep, the informing regulation unit regulates the informing performed by the informing unit. In this manner, even when the biological information is changed due to the change of the state of sleep, it is possible to prevent the load state from being informed. Therefore, the user can sleep in a state of feeling secure.

A second aspect of the invention is directed to a method of controlling a biological information measuring device that measures biological information of a user and informs the user of a load state of the user, including: detecting biological information of the user; detecting motion information related to motion of the user; analyzing the kind of behavior of the user based on the detected biological information and motion information; determining whether informing condition set for the behavior is satisfied based on the detected biological information and motion information; and informing the load state of the user when it is determined that the informing condition is satisfied.

According to the second aspect, it is possible to exhibit the same effects as those of the biological information measuring device according to the first aspect by applying the control method to the biological information measuring device.

A third aspect of the invention is directed to a biological information measuring device including: a biological information detecting unit that detects biological information of a user; a motion information detecting unit that detects motion information related to the motion of the user; an informing unit that informs the user of a load state of the user; an exercise analysis unit that analyzes the kind of exercise of the user based on the detected biological information and motion information; a condition setting unit that sets informing condition of the load state for the kind of exercise; a condition determining unit that determines whether the informing condition is satisfied according to the kind of exercise of the user which is analyzed by the exercise analysis unit based on the detected biological information and motion information; and an informing control unit that informs the informing unit of the load state when it is determined that the informing condition is satisfied by the condition determining unit.

According to the third aspect, the informing condition can be changed according to the type of the current exercise of the user, and thus, informing can be performed based on the informing condition and the current state of exercise of the user. Therefore, the load state of the user (user state in a broad sense) can be effectively informed and the convenience of the biological information measuring device can be improved.

In the third aspect, it is preferable that the kind of exercise includes at least walking and running, and the condition setting unit set at least one of conditions in which the detected biological information exceeds the upper limit of the range of biological information predetermined for the kind of exercise and in which the detected biological information is lower than the lower limit of the range of the biological information as the informing condition in a case where the kind of exercise is one of walking and running.

As the kind of exercise, walking and running can be exemplified.

According to the third aspect with this configuration, in the case where the kind of exercise of the user is walking, the load state is informed when the biological information exceeds the upper limit of the range of the biological information to be set according to walking or when the biological information is lower than the lower limit thereof. Accordingly, for example, in the case where the range of the biological information is the range in which the fat burning efficiency is increased (hereinafter, referred to as a “fat burning zone”), since the load state is informed when the detected biological information exceeds the upper limit thereof or when the detected biological information is lower than the lower limit thereof, it is possible to encourage the user to correct the load state of walking (exercise intensity). That is, it is possible to make the user recognize that the current exercise of the user is not an exercise with high fat burning efficiency.

Further, according to the third aspect, in the case where the kind of exercise of the user is running, the load state is informed when the biological information exceeds the upper limit of the range of the biological information to be set according to running or when the biological information is lower than the lower limit thereof. Therefore, for example, in the case where the range of the biological information is the range to which an aerobic exercise belongs (hereinafter, referred to as an “aerobic exercise zone”), since the load state is informed when the detected biological information exceeds the upper limit thereof or when the detected biological information is lower than the lower limit thereof, it is possible to encourage the user to correct the load state of running (exercise intensity). That is, it is possible to make the user recognize that the current exercise of the user is not an aerobic exercise.

Therefore, it is possible to effectively inform the user of the load state of the user in regard to the exercise.

In the third aspect, it is preferable that running includes high-load running and interval training and the condition setting unit sets the detected biological information exceeding the upper limit of the range of the biological information according to high-load running as the informing condition in a case where the kind of exercise is high-load running and set the detected biological information being lower than the lower limit of the range of the biological information according to interval training as the informing condition in a case where the kind of exercise is interval training.

High-load running means running (sprinting, for example, a last spurt) with relatively high load which is continued for a predetermined time. In addition, interval training means training in which a high-load exercise (running) and a low-load exercise (running) are alternately repeated for a short period of time.

Here, since high-load state is continued at the time of high-load running, the exercise efficiency is not preferable when such a state is continued for a relatively long period of time.

According to the third aspect with this configuration, in the case where the kind of exercise is high-load running, the load state of the user is informed when the detected biological information exceeds the upper limit of the range of the biological information to be set according to high-load running. In this manner, it is possible to encourage the user to correct the load state (exercise intensity) because of continuation of high-load running. That is, it is possible to make the user recognize that the current load state of the user is not appropriate.

Meanwhile, in interval training, since the high-load exercise (running) and the low-load exercise (jogging) are alternately repeated, the pulse rate while the high-load exercise is performed tends to remain at high level in the detected biological information (for example, the pulse rate). Accordingly, the biological information while the low-load exercise is performed has the same level as the pulse rate while the high-load exercise is performed even when the high-load exercise and the low-load exercise are alternately performed. That is, while interval training is performed, when the biological information is lower than the lower limit of the range (aerobic exercise zone) of the biological information to be set according to the kind of exercise (interval training), it is not possible to maximize the beneficial effects of interval training.

According to the third aspect, in the case where the kind of exercise is interval training, the load state of the user is informed when the detected biological information is lower than the lower limit of the range of the biological information to be set according to interval training. Accordingly, it is possible to encourage the user to correct the load state (exercise intensity) of interval training. That is, it is possible to make the user recognize that the current load state of the user is not appropriate.

In the third aspect, it is preferable that running includes normal running other than high-load running and interval training and at least one of threshold values of the upper limit and the lower limit of the range of the biological information according to each of normal running, high-load running, and interval training is higher than the threshold value in the range of the biological information according to walking.

According to the third aspect with this configuration, in the case where the kind of exercise is running, that is, normal running (for example, jogging), high-load running, and interval training, at least one of the upper limit and the lower limit of the range of the biological information is set to be high compared to the case where the kind of exercise is walking. For example, in the case where the kind of exercise is normal running, high-load running, and interval training when informing the load state in the case where the biological information exceeds the upper limit of the range of the biological information to be set according to walking is set as the informing condition, the load state is continuously informed even though the exercise intensity (load state) which is the original purpose is maintained if the upper limit of the range of the biological information to be set according to the kind of exercise is the same as the range of the biological information to be set according to the kind of exercise (walking). This applies to the case where the lower limit of the biological information is lower than the range thereof.

Meanwhile, according to the third aspect, in the case where the kind of exercise is normal running, high-load running, and interval training, since at least one of the upper limit and the lower limit of the range of the biological information is set to be higher compared to the case where the kind of exercise is walking, it is possible to prevent informing that may cause misunderstanding of the load state of the user.

In the third aspect, it is preferable that the biological information measuring device further includes a positional information detecting unit that detects positional information of the user and the exercise analysis unit analyze the kind of exercise of the user based on the biological information, the motion information, and the positional information.

According to the third aspect with this configuration, the kind of exercise of the user is analyzed based on the positional information in addition to the biological information and the motion information of the user. Accordingly, when the change of the positional information is obtained, the moving velocity of the user can be obtained. That is, according to the third aspect, since analysis of the kind of exercise can be performed based on the positional information in addition to the biological information and the motion information, the analysis of the kind of exercise can be more reliably performed.

In the third aspect, it is preferable that the range of the biological information for the kind of exercise is set according to the user.

According to the third aspect with this configuration, since the range of the biological information for the kind of exercise is set based on the user (for example, the maximum pulse rate of the user), the range varies for each user. Consequently, it is possible to set the biological information suitable for both of a user unaccustomed to the exercise and a user accustomed to the exercise such as an athlete.

A fourth aspect of the invention is directed to a method of controlling a biological information measuring device that measures biological information of a user and informs the user of a load state of the user, including: detecting biological information of the user; detecting motion information related to motion of the user; analyzing the kind of exercise of the user based on the detected biological information and motion information; determining whether informing condition set for the kind of exercise is satisfied based on the detected biological information and motion information; and informing the load state of the user when it is determined that the informing condition is satisfied.

According to the fourth aspect, it is possible to exhibit the same effects as those of the biological information measuring device according to the third aspect by applying the control method to the biological information measuring device.

### BRIEF DESCRIPTION OF THE DRAWINGS

The invention will be described with reference to the accompanying drawings, wherein like numbers reference like elements.

FIG. 1 is a perspective view illustrating a biological information measuring device according to a first embodiment of the invention.

FIG. 2 is a block diagram illustrating the configuration of the biological information measuring device according to the embodiment.

FIG. 3 is a block diagram illustrating the configuration of a control unit according to the embodiment.

FIG. 4 is a flowchart illustrating an informing control process according to the embodiment.

FIG. 5 is a diagram illustrating an example of informing a load state in a case where the kind of behavior is exercise (the kind of exercise is walking) according to the embodiment.

FIG. 6 is a diagram illustrating an example of informing the load state in a case where the kind of behavior is non-exercise according to the embodiment.

FIG. 7 is a diagram illustrating an example of informing the load state in a case where the kind of behavior is non-exercise according to a second embodiment of the invention.

FIG. 8 is a block diagram illustrating the configuration of a biological information measuring device according to a third embodiment of the invention.

FIG. 9 is a block diagram illustrating the configuration of a control unit according to the embodiment.

FIG. 10 is a flowchart illustrating an informing control process according to the embodiment.

FIG. 11 is a diagram illustrating an example of informing a load state in a case where the kind of exercise is normal running according to the embodiment.

FIG. 12 is a diagram illustrating an example of informing the load state in a case where the kind of exercise is changed from normal running to high-load running according to the embodiment.

FIG. 13 is a diagram illustrating an example of informing the load state in a case where the kind of exercise is changed from normal running to interval training according to the embodiment.

### DESCRIPTION OF EXEMPLARY EMBODIMENTS

### First Embodiment

### Configuration of Biological Information Measuring Device

Hereinafter, a first embodiment of the invention will be described with reference to the accompanying drawings.

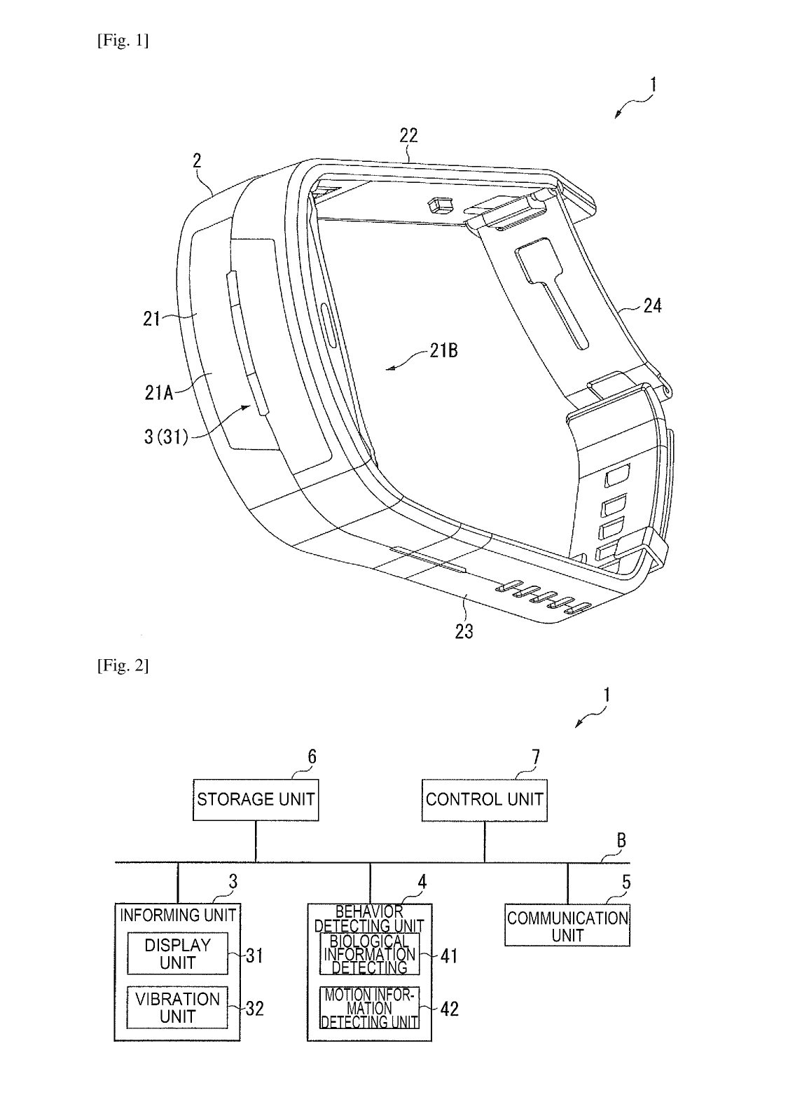

FIG. 1 is a perspective view illustrating a biological information measuring device 1 according to the present embodiment.

The biological information measuring device (hereinafter, also referred to as a “measuring device”) 1 according to the present embodiment detects and measures biological information of a user and motion information accompanied by the motion of the user, analyzes the kind of behavior of the user from the biological information and the motion information, and performs an informing process according to the kind of behavior.

The measuring device 1 is a wristwatch type wearable device that is worn on a wrist of the user and detects and measures the biological information and the motion information. Such a measuring device 1 includes a case 2 constituting the exterior as illustrated in FIG. 1 and the case 2 includes a main body portion 21 and a pair of bands 22 and 23 which are integrally configured respectively, and a buckle 24 that is attached to the bands 22 and 23.

The main body portion 21 is configured to have an approximate arc shape in a side view according to a site at the outside of the wrist (site at the back of the hand). A display unit 31 of an informing unit 3 described below is provided on a front surface portion 21A in the main body portion 21. In addition, a biological information detecting unit 41 of a behavior detecting unit 4 is exposed on a rear surface portion 21B on the opposite side of the front surface portion 21A.

The pair of bands 22 and 23 extend in opposite directions of each other from one end and another end of the main body portion 21 in the longitudinal direction.

The buckle 24 is a middle fastener referred to as a so-called D buckle, which fixes the bands 22 and 23 while the measuring device 1 is worn on the wrist.

In this manner, the measuring device 1 is assumed to be operated by a tapping operation (an operation of tapping the measuring device 1 by hand or fingers) of the user with a purpose of miniaturizing and improving a design property of the measuring device 1, and an operation unit such as a button or the like is not provided on the case 2. However, without limiting thereto, an operation unit such as a button or a touch panel that allows the measuring device 1 to perform a predetermined process may be provided on the case 2.

FIG. 2 is a block diagram illustrating the configuration of the measuring device 1.

As illustrated in FIG. 2, the measuring device 1 includes, in addition to the above-described case 2, an informing unit 3, a behavior detecting unit 4, a communication unit 5, a storage unit 6, and a control unit 7 which are respectively provided in the case 2, and a secondary battery (not illustrated) that supplies power to a constituent component of the measuring device 1. In addition, the informing unit 3, the behavior detecting unit 4, the communication unit 5, the storage unit 6, and the control unit 7 are electrically connected with one another through a bus B.

### Configuration of Informing Unit

The informing unit 3 informs of the operation state and movement state of the measuring device 1 and includes a display unit 31 and a vibration unit 32.

The display unit 31 includes five light emitting diodes (LED) (not illustrated) arranged along the longitudinal direction of the main body portion 21. Further, the display unit 31 displays exercise intensity showing the load state of the user, which is calculated based on detected biological information and acceleration data, according to the tapping operation of the user by lighting (including flickering) or extinguishing the light of respective LEDs under the control of an informing control unit 78 described below.

The vibration unit 32 includes a motor to be controlled by the informing control unit 78 described below and informs the user of the load state (exercise intensity) of the user which is calculated from the biological information and the acceleration data detected from the measuring device 1 through vibration generated due to driving of the motor.

### Configuration of Behavior Detecting Unit

The behavior detecting unit 4 includes the biological information detecting unit 41 that detects biological information of the user and a motion information detecting unit 42 that detects motion information.

The biological information detecting unit 41 is exposed to the rear surface portion 21B and detects the biological information of the user who wears the measuring device 1. In the present embodiment, the biological information detecting unit 41 includes a pulse wave detecting unit that detects the pulse wave as the biological information. The pulse rate (pulse rate per unit time) is measured as the biological information based on the pulse wave detected by the pulse wave detecting unit.

In the present embodiment, as a pulse wave detecting unit, the biological information detecting unit 41 includes a photoelectric sensor including a light emitting element such as an LED and a light receiving element such as a photodiode. However, the biological information detecting unit 41 may include another configuration. For example, the biological information detecting unit 41 may have a configuration that includes an ultrasonic sensor detecting contractions of blood vessels using an ultrasonic wave and measures the pulse rate or a configuration that includes a sensor or a piezoelectric element detecting the pulse by allowing a weak current to flow into a body from an electrode.

The motion information detecting unit 42 detects motion information accompanied with the motion of the user who wears the measuring device 1. In the present embodiment, the motion information detecting unit 42 includes an acceleration sensor that detects an acceleration value (acceleration data) acting on the measuring device 1 according to the motion of the user and outputs the detected acceleration value to the control unit 7. As such an acceleration sensor, a triaxial sensor which detects acceleration values on respective axes of an X axis, a Y axis, and a Z axis using a predetermined sampling frequency (for example, 16 Hz) can be exemplified.

For example, the motion information detecting unit 42 detects the motion of the user and outputs a detected acceleration signal as acceleration data (acceleration value) by amplifying, shaping, and A/D converting the detected acceleration signal in an amplification circuit, a waveform shaping circuit, and an A/D conversion circuit (all not illustrated). The acceleration data for three axes is housed (stored) along the time series in the storage unit 6 as acceleration data rows. The acceleration data stored in this manner is used for behavior analysis of the user and details will be described below.

### Configuration of Communication Unit

The communication unit 5 is a module that communicates with an external device under the control of a movement control unit 72 described below. In the present embodiment, the communication unit 5 is a module that communicates with an external device in a wireless manner (for example, module in conformity with standards of short-distance wireless communication such as IEEE 802.15 standard), but without limiting thereto, the communication unit 5 may be a module that communicates with an external device in a wired manner. Further, the communication unit 5 may be a module that communicates with an external device in a wireless or wired manner.

### Configuration of Storage Unit

The storage unit 6 stores programs and data necessary for movement of the measuring device 1. As such programs, the storage unit 6 stores an informing control program that allows the control unit 7 to perform an informing control process described below.

Further, the storage unit 6 stores detection results by the behavior detecting unit 4 under the control of the control unit 7 and stores informing condition set by a condition setting unit 74 described below and used for the informing control process. Such a storage unit 6 can be configured of a non-volatile semiconductor memory such as a flash memory.

### Configuration of Control Unit

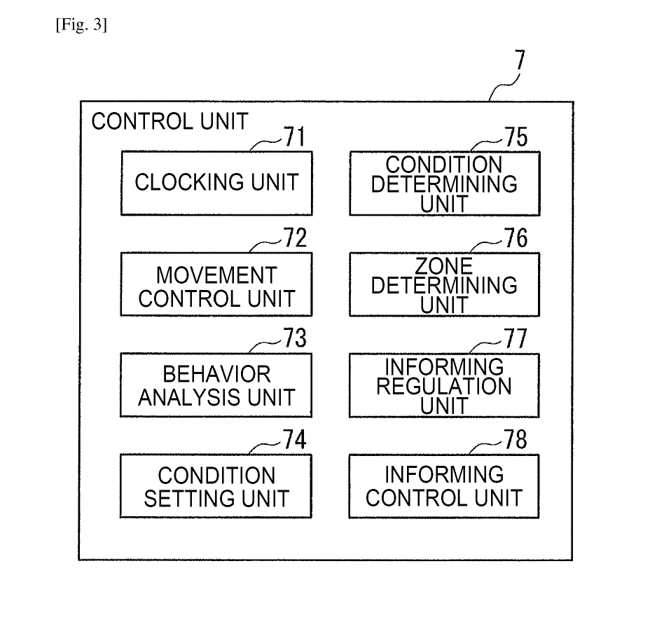

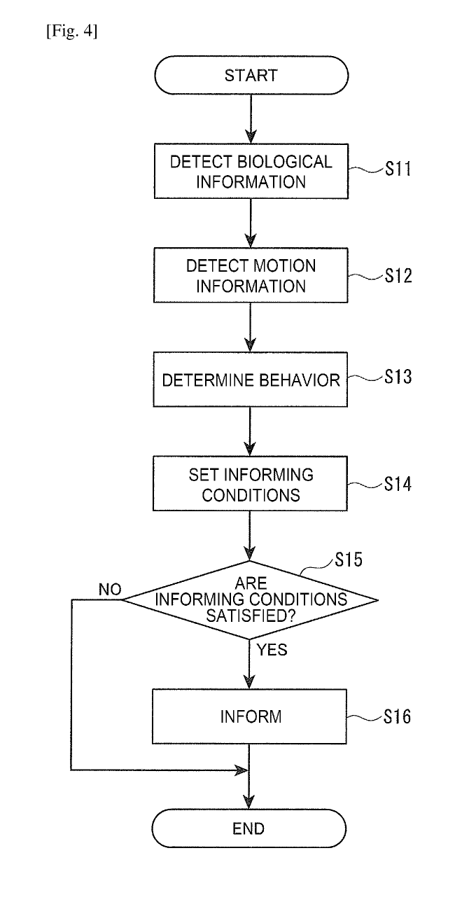

FIG. 3 is a block diagram illustrating the configuration of the control unit 7.

The control unit 7 is configured of a control circuit and controls the movement of the measuring device 1. That is, the control unit 7 autonomously controls the movement of the measuring device 1. In addition, the control unit 7 performs an informing control process that informs the user of the load state of the user in the case where informing conditions are satisfied based on the biological information and the motion information which are detected by the behavior detecting unit 4.

As illustrated in FIG. 3, such a control unit 7 includes a clocking unit 71, the movement control unit 72, the behavior analysis unit 73, the condition setting unit 74, a condition determining unit 75, a zone determining unit 76, an informing regulation unit 77, and the informing control unit 78 as a function unit that realizes respective programs stored in the storage unit 6 by the above-described control circuit performing the programs.

The clocking unit 71 clocks the current date and time.

The movement control unit 72 controls the movement of the entire device. For example, the movement control unit 72 measures the pulse rate by analyzing the pulse wave detected by the biological information detecting unit 41, stores the pulse rate in the storage unit 6, and stores the acceleration data detected by the motion information detecting unit 42 in the storage unit 6. Moreover, the movement control unit 72 determines operation contents of the user based on a change pattern of acceleration detected by the motion information detecting unit 42 and performs a process according to the operation contents. For example, the movement control unit 72 determines that a tapping operation is performed in a case where the acceleration value according to continuous impacts due to the tapping operation with respect to the measuring device 1 is changed and performs a process according to an operation mode.

The behavior analysis unit 73 analyzes the kind of behavior of the user based on the pulse rate as the biological information and the acceleration data as the motion information stored in the storage unit 6 respectively detected by the biological information detecting unit 41 and the motion information detecting unit 42.

Here, as the kind of behavior, exercise, non-exercise, or sleep can be exemplified.

For example, the behavior analysis unit 73 analyzes the kind of behavior of the user based on the difference of acceleration, the number of occurrences of the difference, a determination value (threshold value), and the pulse rate in an index table set for the kind of behavior stored in the storage unit 6. That is, the behavior of the user is analyzed as exercise when the change amount of the acceleration is large and the pulse rate is high and the kind of behavior of the user is determined as non-exercise when the change amount of the acceleration is small and the pulse rate is low. Further, in regard to sleep included in non-exercise in the kind of behavior, it is analyzed whether the behavior is sleep in non-exercise based on a change of the pulse rate in REM sleep and non-REM sleep.

The condition setting unit 74 sets informing conditions of whether to inform the user of the load state of the user for the kind of behavior and stores the informing conditions in the storage unit 6.

Here, the load state of the user acquired from the biological information (pulse rate) will be described.

In the present embodiment, in a case where the kind of behavior to be analyzed based on the pulse rate and the acceleration data is exercise, a fat burning zone which is a range (range of the pulse rate in which fat burning efficiency is increased) of the pulse rate, in which body fat can be effectively burned when the user does an exercise with the exercise intensity within the range, is set for each user. At this time, the fat burning zone is set in a range of 40% to less than 70% of exercise intensity (Heart Rate Reserve) to be acquired by a Karvonen method. Further, the zone may be set based on various pieces of information such as the height, weight, age, sex, and presence or absence of exercise experience of the user in consideration of individual differences in an increase or decrease tendency of the pulse rate.

Moreover, without limiting thereto, such a zone may be set based on the exercise intensity to be acquired by a Mafeton method and the zone may be set as a range (for example, a range of 40% to 70% of the maximum pulse rate of the user) indicated by the upper limit and the lower limit acquired by multiplying the maximum pulse rate of the user by a predetermined ratio (proportion).

Further, when the kind of behavior is exercise, the condition setting unit 74 sets a case where the biological information (pulse rate) is within the fat burning zone, a case where the biological information exceeds the upper limit of the fat burning zone, and a case where the biological information is lower than the lower limit of the fat burning zone as informing conditions of informing the user of the load state at the time of exercise.

In addition, when the kind of behavior of the user is non-exercise, the condition setting unit 74 sets a case where the biological information exceeds the threshold value stored in the storage unit 6 in advance as the informing condition of informing the user of the load state. The threshold value is set as the lower limit of the fat burning zone according to the present embodiment. The condition setting unit 74 regulates setting of other cases (for example, cases where the biological information is lower than the threshold value) as the informing conditions of the load state.

Further, the informing conditions set by the condition setting unit 74 are not limited thereto. For example, informing conditions can be arbitrarily set by the user.

The condition determining unit 75 determines whether to inform the user of the load state based on the kind of behavior of the user analyzed by the behavior analysis unit 73, that is, the kind of current behavior of the user, and conditions stored in the storage unit 6 according to the kind of behavior. Specifically, the condition determining unit 75 obtains the informing conditions according to the kind of current analyzed behavior of the user with reference to the storage unit 6 and determines whether the informing conditions are satisfied based on the detected biological information and motion information.

The zone determining unit 76 determines whether the biological information obtained by the biological information detecting unit 41 is within the fat burning zone, the biological information exceeds the upper limit, or the biological information is lower than the lower limit. According to the determination results of the zone determining unit 76, the number of lighting of an LED of the display unit 31 during the tapping operation is changed by the informing control unit 78.

The informing regulation unit 77 regulates informing the user of the load state by the informing unit 3 in the case where the kind of analyzed behavior of the user is sleep which is included in non-exercise.

When the tapping operation is performed, the informing control unit 78 displays the exercise intensity (load state) and the number of tapping using the number of lighting of the LED of the display unit 31.

Further, the informing control unit 78 informs the user of the load state by controlling the vibration unit 32 so that the measuring device 1 is vibrated when the condition determining unit 75 determines that the informing conditions according to the kind of current behavior are satisfied.

### Informing Control Process

FIG. 4 is a flowchart illustrating an informing control process performed by the control unit 7.

When the user wears the measuring device 1, the control unit 7 performs the informing control process described below based on the programs stored in the storage unit 6.

In the informing control process, as illustrated in FIG. 4, first, the movement control unit 72 operates the behavior detecting unit 4 and detects the biological information and the motion information (acceleration) of the user using the biological information detecting unit 41 and the motion information detecting unit 42 constituting the behavior detecting unit 4 (Steps S11 and S12). Detections of the biological information and the motion information are at least continued by the respective detecting units 41 and 42 while the measuring device is worn on the user, and the detected biological information and motion information are stored in the storage unit 6 as described above.

Further, the behavior analysis unit 73 analyzes the kind of current behavior (behavior kind) of the user based on the biological information and the motion information which are stored in the storage unit 6 (Step S13).

Subsequently, the condition setting unit 74 sets the informing conditions based on the kind of behavior analyzed by the behavior analysis unit 73 (Step S14).

Further, the condition determining unit 75 determines whether the set informing conditions are satisfied (Step S15).

In the determination process in Step S15, when it is determined that the informing conditions are not satisfied, the informing control process is terminated without informing the user by the informing unit 3. Further, the process is terminated in the flowchart in FIG. 4, but the informing control process is repeatedly performed. For example, the control unit 7 returns the process to Step S11 after the process in Step S15 is performed and the above-described process is repeated.

In contrast, when it is determined that the informing conditions are satisfied in the determination process in Step S15, the informing control unit 78 allows the informing unit 3 to inform the user of the load state (Step S16). Specifically, the informing control unit 78 allows the informing unit 3 to inform the user of the load state by allowing the vibration unit 32 to be driven so that the measuring device 1 is vibrated. Moreover, the control unit 7 terminates the informing control process.

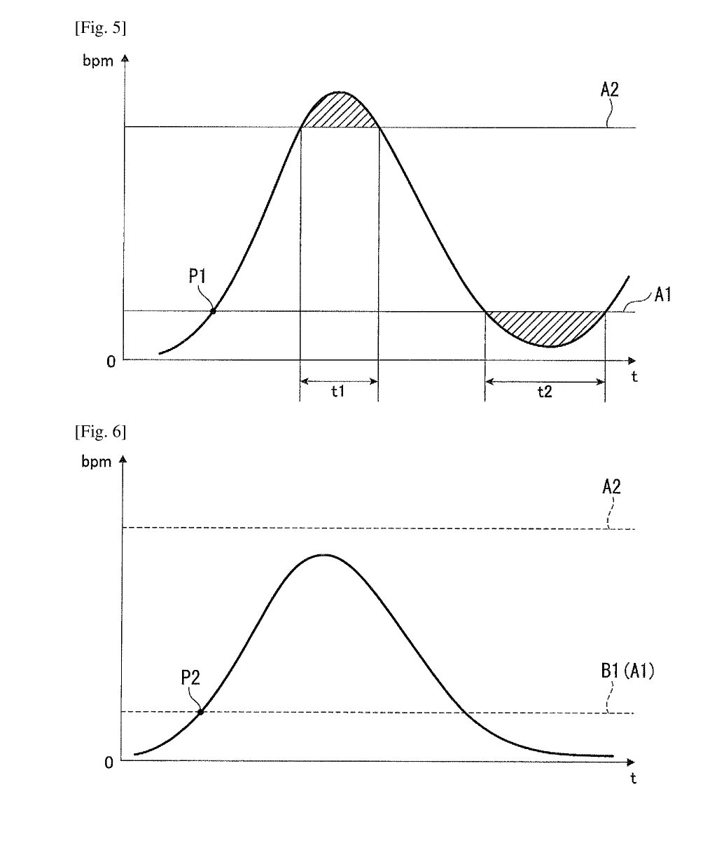

FIG. 5 is a diagram illustrating an example of informing in the case where the kind of behavior is exercise (the kind of exercise is walking) and FIG. 6 is a diagram illustrating an example of informing in the case where the kind of behavior is non-exercise. Further, a straight line A1 in FIG. 5 indicates the lower limit of the fat burning zone and a straight line A2 indicates the upper limit. Further, a dotted line B1 in FIG. 6 indicates a threshold value referenced by the informing determination and the dotted line B1 coincides with the straight line A1 since the threshold value is set to be the same as the lower limit of the fat burning zone as described above. Further, a dotted line A2 in FIG. 6 indicates the upper limit of the fat burning zone.

In the case where the kind of behavior determined in Step S13 is exercise, the informing control unit 78 informs of the time, the period, and the load state in which the informing conditions set by the condition setting unit 74 are satisfied.

For example, as illustrated in FIG. 5, the informing control unit 78 allows the informing unit 3 to inform the user at a time point P1 when the pulse rate exceeds a lower limit A1 of the fat burning zone and the informing unit 3 informs the user of exercise belonging to the fat burning zone being performed.

Moreover, after the pulse rate is in the fat burning zone, the informing control unit 78 allows the informing unit 3 to inform the user and the informing unit 3 informs the user of exercise with extreme exercise intensity being performed during a period t1 when the pulse rate exceeds an upper limit A2 of the fat burning zone. At this time, for example, the informing control unit 78 may immediately suppress exercise by allowing the vibration unit 32 to be driven such that a cycle of vibration being generated becomes relatively short or may calm the user by allowing the vibration unit 32 to be driven such that the cycle becomes relatively long.

Further, after the pulse rate is in the fat burning zone, the informing control unit 78 allows the informing unit 3 to inform the user and the informing unit 3 informs the user of exercise not reaching the fat burning zone being performed during a period t2 when the pulse rate is lower than a lower limit A1 of the fat burning zone. At this time, for example, the informing control unit 78 may encourage the user to exercise with high exercise intensity by allowing the vibration unit 32 to be differently driven from the cycle of vibration in the case where the cycle of vibration being generated exceeds the upper limit.

Moreover, in the case where the kind of behavior determined in Step S13 is non-exercise, the informing control unit 78 allows the informing unit to inform the user of the load state when the informing conditions set by the condition setting unit 74 are satisfied. For example, as illustrated in FIG. 6, the informing control unit 78 allows the informing unit 3 to inform the user at a time point P2 when the pulse rate exceeds a threshold value B1 (same value as the lower limit A1 of the fat burning zone).

In addition, in the case where the kind of behavior is non-exercise, since the informing unit 3 informs the user only when it is determined that the biological information (pulse rate) exceeds the threshold value B1, the informing unit 3 does not inform the user when the biological information (pulse rate) exceeds the upper limit A2 of the fat burning zone and the biological information is lower than the threshold value B1.

Further, in the case where the kind of behavior determined in Step S13 is sleep included in non-exercise, informing using the informing unit 3 is regulated by the informing regulation unit 77.

### Effects of First Embodiment

According to the biological information measuring device 1 of the present embodiment described above, there are following effects.

According to the biological information measuring device 1 of the present embodiment, the informing conditions according to the kind of behavior of the user analyzed based on the biological information (pulse rate) and the acceleration data detected by the behavior detecting unit 4 are set by the condition setting unit 74, and informing determination of the load state of the user is performed by the condition determining unit 75 based on the informing conditions and the informing unit 3 informs the user of the load state of the user in the case where it is determined that the conditions are satisfied.

According to this, since the informing conditions are set according to the kind of current behavior of the user and the load state is informed when it is determined that the informing conditions are satisfied, informing according to the kind of current behavior of the user can be performed. Therefore, the load state of the user can be effectively informed and the convenience of the biological information measuring device can be improved.

According to the present embodiment, in the case where the kind of behavior of the user is at least one of exercise and non-exercise, the load state of the user is informed when the detected biological information exceeds the lower limit of the range of the biological information to be set according to the user.

In this manner, in the case where the kind of behavior of the user is exercise and the detected biological information exceeds the lower limit A1 of the fat burning zone, it is possible to encourage the user to maintain the behavior when the load state of the user is informed. Therefore, it is possible to allow the user to maintain the state with high fat burning efficiency.

According to the present embodiment, in the case where the kind of behavior is at least one of exercise and non-exercise, the load state of the user is informed when the detected biological information exceeds the upper limit A2 of the fat burning zone to be set according to the user. In this manner, for example, in the case where the kind of behavior of the user is exercise, it is possible to encourage the user to correct the behavior when the load state is informed. That is, it is possible to allow the user to recognize that the current exercise intensity of the user is not appropriate and the current exercise is not an exercise with high fat burning efficiency.

According to the present embodiment, in the case where the kind of behavior is exercise, the load state of the user is informed when the detected biological information is lower than the lower limit A1 of the fat burning zone. In this manner, when the user is informed of the fact that the biological information is lower than the lower limit A1 of the fat burning zone, it is possible to encourage the user to correct the behavior. That is, it is possible to allow the user to recognize that the current exercise intensity of the user is not appropriate and the current exercise is an exercise with low fat burning efficiency.

Meanwhile, in the case where the kind of behavior of the user is non-exercise, when the detected biological information is lower than the threshold value B1 which is the same as the lower limit A1 of the fat burning zone, informing the user of the load state is regulated. In this manner, the psychological load of the user is alleviated, and the load state is not informed when the user is calm. Accordingly, it is possible to prevent the load state from being increased by the user being excited again or the like and to maintain a stress-free state.

According to the present embodiment, in the case where it is determined that the kind of behavior of the user is sleep, informing the user of the load state by the informing unit 3 is regulated by the informing regulation unit 77. In this manner, even when the biological information is changed due to the change of the state of sleep, it is possible to prevent the load state from being informed. Therefore, the user can sleep in a state of feeling secure.

### Second Embodiment

Next, a second embodiment of the invention will be described.

A biological information measuring device according to the present embodiment has the configuration which is the same as that of the above-described biological information measuring device 1, and the condition setting unit 74 has a function of setting a zone different from the fat burning zone in the case where the kind of behavior of the user is non-exercise. Further, the informing conditions of the load state in the case where the kind of behavior to be set by the condition setting unit 74 are different from those of the above-described biological information measuring device 1. In these terms, the biological information measuring device according to the present embodiment is different from the above-described biological information measuring device. Further, in the description below, units which are the same or substantially the same as the units described above are denoted by the same reference numerals and the description thereof is not repeated.

The condition setting unit 74 changes the zone into a zone (hereinafter, referred to as a “stress zone” in some cases) whose upper limit (upper limit A2) is lower than the above-described fat burning zone set according to the user when the kind of behavior is exercise in the case where the behavior analysis unit 73 determines that the kind of behavior of the user is non-exercise.

The upper limit of such a stress zone can be set to be in the range of 20% to 50% of the maximum pulse rate in a case where the fat burning zone is set to be in the range of 40% to 70% of the maximum pulse rate when the kind of behavior is exercise.

Further, according to the present embodiment, in the case where it is determined that the kind of behavior is non-exercise, the condition setting unit 74 applies not the fat burning zone to be applied when the kind of behavior is exercise but the stress zone.

Moreover, in the case where the kind of behavior of the user is non-exercise, the condition setting unit 74 sets a case where the biological information (pulse rate) exceeds the lower limit of the stress zone and a case where the biological information exceeds the upper limit of the stress zone as the informing conditions of informing the user of the load state and regulates setting other cases (for example, the biological information is lower than the lower limit of the stress zone) as the informing conditions.

The informing control process to be performed by the measuring device of the present embodiment is performed in the same manner as the informing control process to be performed by the above-described measuring device 1 by following procedures illustrated in FIG. 4.

That is, when it is determined that the kind of behavior is non-exercise in Step S13 and it is determined that the informing conditions set in Step S14 are satisfied in Step S15, the informing control unit 78 allows the informing unit 3 to inform the user of the load state as described above.

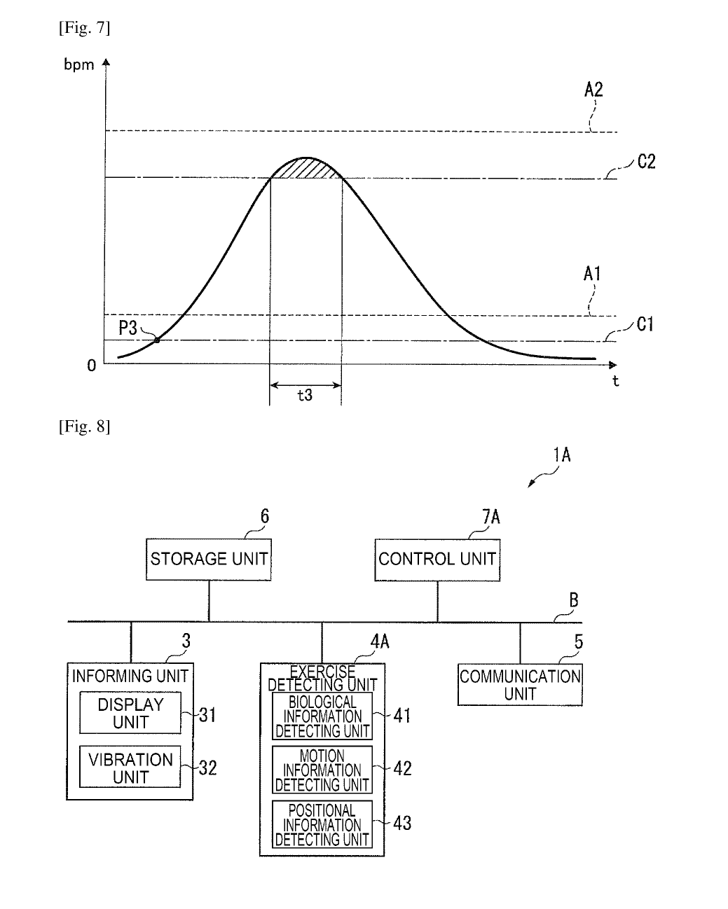

FIG. 7 is a diagram illustrating an example of informing in the case where the kind of behavior is non-exercise according to the present embodiment. A dotted line A1 in FIG. 7 indicates the lower limit of the fat burning zone and a dotted line A2 indicates the upper limit thereof. In addition, a dashed line C1 indicates the lower limit of the stress zone and a dashed line C2 indicates the upper limit of the stress zone.

For example, as illustrated in FIG. 7, the informing control unit 78 allows the informing unit 3 to inform the user at a time point P3 when the pulse rate exceeds a lower limit C1 of the stress zone. Moreover, after the pulse rate is in the stress zone, the informing control unit 78 allows the informing unit 3 to inform the user during a period t3 when the pulse rate exceeds an upper limit C2 of the stress zone.

In addition, in the case where the kind of behavior is non-exercise, since setting the case where the biological information is lower than the lower limit C1 of the stress zone after the biological information is in the stress zone as the informing condition of the load state is regulated, the informing unit 3 does not inform the user.

Moreover, as described above, informing using the informing unit 3 is regulated by the informing regulation unit 77 even in the case where the kind of behavior is sleep which is included in non-exercise.

### Effects of Second Embodiment

Effects of the biological information measuring device of the second embodiment are the same as those of the biological information measuring device 1 of the first embodiment, and the following effects are exhibited.

According to the present embodiment, in the case where the kind of behavior is exercise or non-exercise, the load state of the user is informed when the detected biological information exceeds the lower limits A1 and C1 of the range (fat burning zone and the stress zone) of the biological information to be set according to the user.

In this manner, for example, in the case where the kind of behavior of the user is exercise, it is possible to encourage the user to maintain the behavior when the load state is informed and to encourage the user to maintain the state with high fat burning efficiency.

Further, in the case where the kind of behavior of the user is non-exercise and the load state is informed, the user can recognize that he or she has a psychological load, that is, he or she is under stress, so that the user can objectively recognize the factor causing the stress. Moreover, since there is a possibility that concentration of the user can be improved and greater ability than usual can be exhibited when the user is under a moderate degree of stress, the user can try to maintain the moderate degree of stress.

Therefore, the load state can be effectively informed in both cases where the kind of behavior of the user is exercise and the kind of behavior of the user is non-exercise.

According to the present embodiment, in the case where the kind of behavior is exercise or non-exercise, the load state of the user is informed when the detected biological information exceeds the upper limits A2 and C2 of the fat burning zone or the stress zone to be set according to the user. In this manner, for example, in the case where the kind of behavior of the user is exercise, it is possible to encourage the user to correct the behavior when the load state is informed. That is, it is possible to allow the user to recognize that the current exercise intensity of the user is not appropriate and the current exercise is not an exercise with high fat burning efficiency.

Meanwhile, in the case where the kind of behavior of the user is non-exercise and the load state is informed, the user can recognize that he or she has a psychological load which exceeds the upper limit, that is, he or she is under extreme stress. Accordingly, it is possible to encourage the user to eliminate the factor of the stress.

Therefore, the load state can be effectively informed in both cases where the kind of behavior of the user is exercise and the kind of behavior of the user is non-exercise.

According to the present embodiment, in the case where the kind of behavior of the user is non-exercise, informing the user of the load state is regulated when the detected biological information is lower than the lower limit C1 of the stress zone. In this manner, the psychological load of the user is alleviated, and the load state is not informed when the user is calm. Accordingly, it is possible to maintain a stress-free state.

According to the present embodiment, in the case where the kind of behavior is non-exercise, the upper limit of the range of the biological information to be set according to the user is set as the upper limit C2 which is lower than the upper limit A2 of the fat burning zone. That is, the change of the biological information at the time of non-exercise, whose width of the change of the biological information is smaller than that at the time of exercise, can be reliably detected by setting the stress zone. Therefore, the load state of the user can be accurately informed.

### Third Embodiment

Next, a third embodiment of the invention will be described.

A biological information measuring device according to the present embodiment includes the configuration which is the same as that of the above-described biological information measuring device 1. Here, the biological information measuring device includes an exercise detecting unit in place of the behavior detecting unit 4. Further, the configuration of a control unit is different from that of the control unit 7. Specifically, the control unit includes an exercise analysis unit in place of the behavior analysis unit 73. Further, the control unit does not include the informing regulation unit 77. In these terms, the biological information measuring device according to the present embodiment is different from the above-described biological information measuring device 1.

Further, in the description below, units which are the same or substantially the same as the units described above are denoted by the same reference numerals and the description thereof is not repeated.

FIG. 8 is a block diagram illustrating the configuration of a biological information measuring device 1A.

The biological information measuring device (hereinafter, referred to as a “measuring device 1A” in some cases) 1A as illustrated in FIG. 8 includes the informing unit 3, an exercise detecting unit 4A, the communication unit 5, the storage unit 6, and a control unit 7A.

The exercise detecting unit 4A includes the biological information detecting unit 41, the motion information detecting unit 42, and a positional information detecting unit 43 that detects positional information of the user.

The positional information detecting unit 43 detects the positional information of the user who wears the measuring device 1A. In the present embodiment, the positional information detecting unit 43 includes a receiving device that receives a satellite signal sent from a positional information satellite (for example, a GPS satellite and a quasi-zenith satellite) and a positional information calculating device (not illustrated) that calculates the positional information of the user from the satellite signal received from the receiving device, and outputs the positional information acquired from these to the control unit 7A.

### Configuration of Control Unit

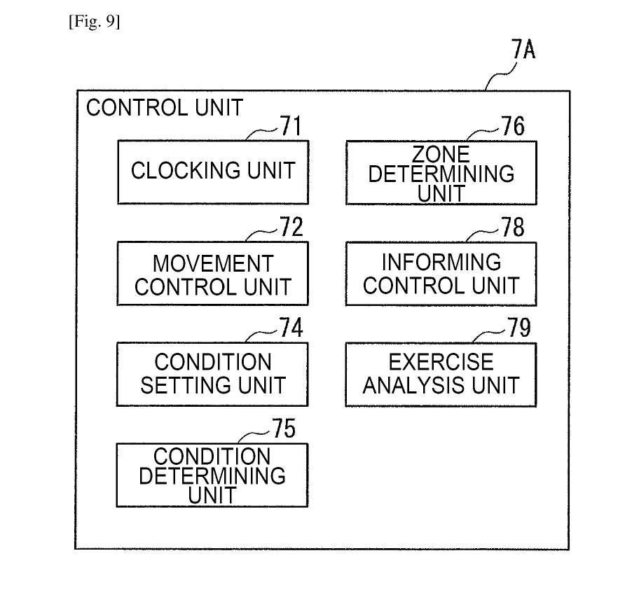

FIG. 9 is a block diagram illustrating the configuration of the control unit 7A.

The control unit 7A is configured of a control circuit and controls the movement of the measuring device 1A. That is, the control unit 7A autonomously controls the movement of the measuring device 1A. In addition, the control unit 7A performs an informing control process that informs the user of the load state of the user in the case where informing conditions are satisfied based on the biological information, the motion information, and the positional information which are detected by the exercise detecting unit 4A.

As illustrated in FIG. 9, such a control unit 7A includes the clocking unit 71, the movement control unit 72, the condition setting unit 74, the condition determining unit 75, the zone determining unit 76, the informing control unit 78, and an exercise analysis unit 79 as a function unit that realizes respective programs stored in the storage unit 6 by the above-described control circuit performing the programs.

The movement control unit 72 controls the operation of the entire device. For example, the movement control unit 72 measures the pulse rate, stores the pulse rate in the storage unit 6, and stores the acceleration data detected by the motion information detecting unit 42 and the positional information acquired by the positional information detecting unit 43 in the storage unit 6.

The exercise analysis unit 79 analyzes the kind of exercise of the user based on the pulse rate as the biological information, the acceleration data as the motion information, and the positional information stored in the storage unit 6 respectively detected by the biological information detecting unit 41, the motion information detecting unit 42, and the positional information detecting unit 43.

Here, as the kind of exercise, walking or running (normal running, high-load running, and interval training) can be exemplified.

Further, according to the present embodiment, normal running means jogging or running with a small change in velocity, high-load running means sprinting (last spurt) performed at the end of running, and interval training means exercise in which running with relatively high load and running (jogging) with relatively low load are alternately repeated for a short period of time.

For example, the exercise analysis unit 79 analyzes the kind of exercise of the user based on the difference of acceleration, the number of occurrences of the difference, a determination value (threshold value), the pulse rate, and the positional information (moving velocity to be calculated from the positional information) in an index table set for the kind of exercise stored in the storage unit 6. That is, when the change amount of the acceleration is an average, the pulse rate is an average, and the moving velocity is approximately in the range of 2 km to 6 km per hour, the kind of exercise of the user is analyzed as walking. Further, when the change amount of the acceleration is high, the pulse rate is high, and the moving velocity is approximately in the range of 6 km to 19 km per hour, the kind of exercise of the user is analyzed as running. Further, when the change amount of the acceleration is high, the pulse rate is extremely high, and the moving velocity is approximately in the range of 20 km to 30 km per hour, the kind of exercise of the user is analyzed as high-load running. In addition, when the change amount of acceleration and the moving velocity are alternately repeated with a certain period and the pulse rate is maintained to be high, the kind of exercise of the user is analyzed as interval training.

That is, in the analysis of interval training, first, when high-load running and low-load running are alternately repeated for a short period of time after the kind of exercise is determined as normal running, the kind of exercise is analyzed as interval training.

The condition setting unit 74 sets the informing conditions determining whether to inform the user of the load state for each kind of exercise, and stores the informing conditions in the storage unit 6.

Here, the load state (user state in a broad sense) of the user acquired from the biological information (pulse rate) will be described.

In the present embodiment, in a case where the kind of exercise to be analyzed based on the pulse rate, the acceleration data, and the positional information is walking, a fat burning zone which is a range (range of the pulse rate in which fat burning efficiency is increased) of the pulse rate, in which body fat can be effectively burned when the user does an exercise with the exercise intensity within the range, is set for each user. At this time, the fat burning zone is set in a range of 40% to less than 70% of exercise intensity (Heart Rate Reserve) to be acquired by a Karvonen method. Further, the fat burning zone may be set based on various pieces of information such as the height, weight, age, sex, and presence or absence of exercise experience of the user in consideration of individual differences in an increase or decrease tendency of the pulse rate.

In the case where the kind of exercise to be analyzed based on the pulse rate, the acceleration data, and the positional information is running, high-load running, or interval training, an aerobic exercise zone which is a range of the pulse rate in which effective aerobic exercise can be performed when the user exercises with the exercise intensity within the range is set for each user. At this time, the aerobic exercise zone is set in a range of 70% to less than 80% of exercise intensity to be acquired by the Karvonen method. Further, the aerobic exercise zone may be set based on various pieces of information such as the height and presence or absence of exercise experience of the user in the same manner as that of the above-described fat burning zone.

Further, when the kind of exercise is walking, the condition setting unit 74 sets a case where the biological information (pulse rate) is within the fat burning zone, a case where the biological information exceeds the upper limit of the fat burning zone, and a case where the biological information is lower than the lower limit of the fat burning zone as informing conditions of informing the user of the load state at the time of walking.

Further, when the kind of exercise is normal running, the condition setting unit 74 sets a case where the pulse rate is in the aerobic exercise zone, a case where the biological information exceeds the upper limit of the aerobic exercise zone, and a case where the biological information is lower than the lower limit of the aerobic exercise zone as informing conditions of informing the user of the load state at the time of normal running.

Further, when the kind of exercise is high-load running, the condition setting unit 74 sets a case where the pulse rate exceeds the upper limit of the aerobic exercise zone as the informing condition of informing the user of the load state at the time of high-load running. In addition, when the kind of exercise is interval training, the condition setting unit 74 sets a case where the pulse rate is lower than the lower limit of the aerobic exercise zone as the informing condition of informing the user of the load state at the time of interval training.

Moreover, without limiting thereto, the informing conditions set by the condition setting unit 74 can be arbitrarily set by the user.

The condition determining unit 75 determines whether to inform the user of the load state based on the kind of exercise of the user analyzed by the exercise analysis unit 79, that is, the kind of current exercise of the user, and conditions stored in the storage unit 6 according to the kind of exercise. Specifically, the condition determining unit 75 obtains the informing conditions according to the kind of current analyzed exercise of the user with reference to the storage unit 6 and determines whether the informing conditions are satisfied based on the detected biological information, motion information, and positional information.

The zone determining unit 76 determines whether the biological information obtained by the biological information detecting unit 41 belongs to the fat burning zone, the biological information exceeds the upper limit of the zone, or the biological information is lower than the lower limit of the zone. Further, the zone determining unit 76 determines whether the biological information obtained by the biological information detecting unit 41 belongs to the aerobic exercise zone, the biological information exceeds the upper limit of the zone, or the biological information is lower than the lower limit of the zone. According to the determination results of the zone determining unit 76, the number of lighting of an LED of the display unit 31 during the tapping operation is changed by the informing control unit 78.

### Informing Control Process

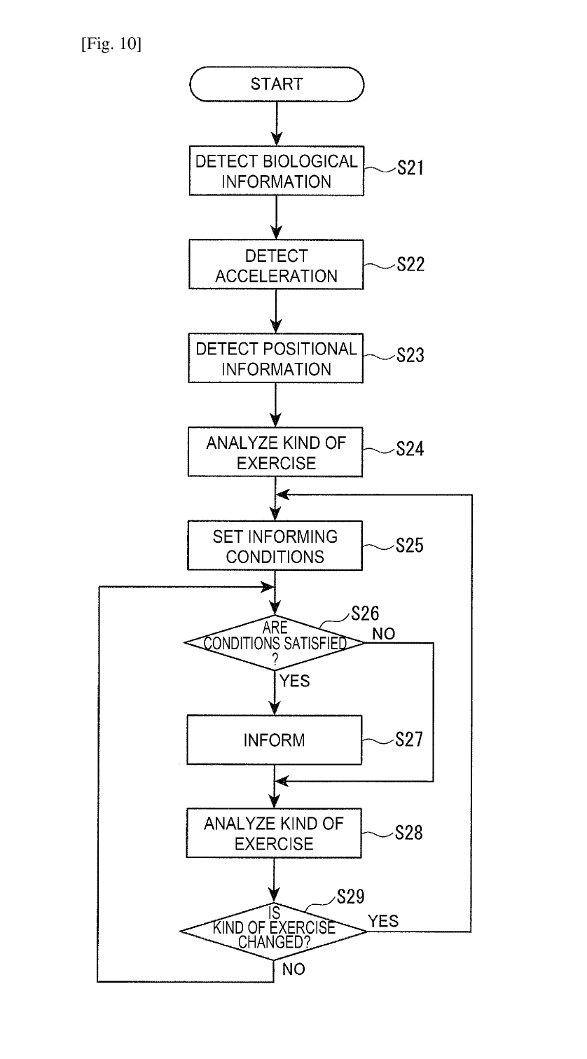

FIG. 10 is a flowchart illustrating the informing control process performed by the control unit 7A.

When the user wears the measuring device 1A, the control unit 7A performs the informing control process described below based on the programs stored in the storage unit 6.

In the informing control process, as illustrated in FIG. 10, first, the movement control unit 72 operates the exercise detecting unit 4A and detects the biological information, the motion information, and the positional information of the user using the biological information detecting unit 41, the motion information detecting unit 42, and the positional information detecting unit 43 constituting the exercise detecting unit 4A (Steps S21, S22, and S23). Detection of the biological information, the motion information, and the positional information is continued by the respective detecting units 41, 42, and 43 using sampling rates to be respectively and individually set while the measuring device is at least worn on the user, and the detected biological information, the motion information, and the positional information are stored in the storage unit 6 as described above.

Further, the exercise analysis unit 79 analyzes the kind of current exercise (exercise kind) of the user based on the biological information, the motion information, and the positional information (moving velocity) which are stored in the storage unit (Step S24). At this time point of Step S24, it is determined whether the kind of current exercise is one of walking and running (normal running).

Subsequently, the condition setting unit 74 sets the informing conditions based on the kind of exercise analyzed by the exercise analysis unit 79 (Step S25).

Further, the condition determining unit 75 determines whether the set informing conditions are satisfied (Step S26).

When it is determined that the informing conditions are satisfied during the determining process in Step S26, the informing control unit 78 allows the informing unit 3 to inform the user of the load state (exercise intensity) (Step S27). Specifically, the informing control unit 78 allows the informing unit 3 to inform the user of the load state by allowing the vibration unit 32 to be driven so that the measuring device 1A is vibrated.

In the determination process in Step S26, analysis of the kind of exercise is performed again by the exercise analysis unit 79 after it is determined that the informing conditions are not satisfied and the exercise intensity is informed in Step S27 described above (Step S28). Further, in Step S29, it is determined whether the kind of the exercise analyzed in Step S24 is different from the kind of exercise analyzed in Step S28 (Step S29). In this manner, when it is determined that the kinds of exercise are not different from each other, that is, it is determined that the kinds of exercise are not changed, the control unit 7 returns the process to Step S26 and the above-described processes are repeated.

For example, in the case where the kind of exercise is analyzed as walking in Step S24 and the kind of exercise is not changed, the informing conditions are not changed and the process of determining whether the same conditions are satisfied is repeatedly performed.

Meanwhile, in Step S29, when it is determined that the kind of exercise analyzed in Step S24 is different from the kind of exercise analyzed in Step S28, that is, it is determined that the kinds of exercise are changed, the control unit 7A returns the process to Step S25, sets the informing conditions based on the kind of changed exercise, and repeats the above-described process.

For example, when the kind of exercise is analyzed as normal running in Step S24 and the kind of exercise is analyzed as interval training in Step S29, the control unit 7A returns the process to Step S25, sets the informing conditions in accordance with interval training, and repeatedly performs the process of determining whether the set conditions are satisfied.

### Informing Example for Each Kind of Exercise

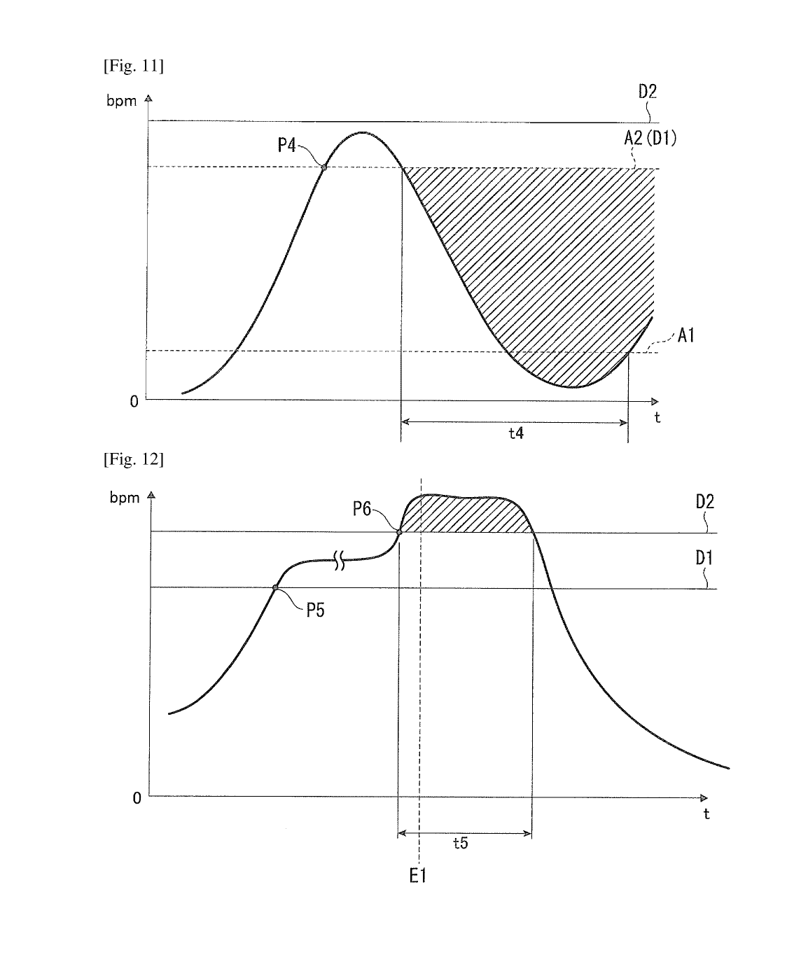

FIG. 5 is a diagram illustrating an informing example in the case where the kind of exercise is walking. FIG. 11 is a diagram illustrating an informing example in the case where the kind of exercise is normal running. Moreover, the straight line A1 in FIG. 5 indicates the lower limit of the fat burning zone and the straight line A2 indicates the upper limit. Further, a straight line D1 in FIG. 11 indicates the lower limit of the aerobic exercise zone and a straight line D1 coincides with the straight line A2 since the straight line D1 is set to be the same value as the upper limit of the fat burning zone as described above. Further, a straight line D2 in FIG. 11 indicates the upper limit of the aerobic exercise zone.

### Case of Walking

In the case where the kind of exercise determined in Step S24 is walking, the informing control unit 78 informs of the time, the period, and the load state in which the informing conditions set by the condition setting unit 74 are satisfied.

For example, as illustrated in FIG. 5, the informing control unit 78 allows the informing unit 3 to inform the user at the time point P1 when the pulse rate exceeds the lower limit A1 of the fat burning zone and the informing unit 3 informs the user of exercise belonging to the fat burning zone being performed as described below.

Moreover, after the pulse rate is in the fat burning zone, the informing control unit 78 allows the informing unit 3 to inform the user and the informing unit 3 informs the user of exercise with extreme exercise intensity being performed during the period t1 when the pulse rate exceeds the upper limit A2 of the fat burning zone. At this time, for example, the informing control unit 78 may immediately suppress exercise by allowing the vibration unit 32 to be driven such that a cycle of vibration being generated becomes relatively short or may calm the user by allowing the vibration unit 32 to be driven such that the cycle becomes relatively long.

Further, after the pulse rate is in the fat burning zone, the informing control unit 78 allows the informing unit 3 to inform the user and the informing unit 3 informs the user of exercise not reaching the fat burning zone being performed during the period t2 when the pulse rate is lower than a lower limit A1 of the fat burning zone. At this time, for example, the informing control unit 78 may encourage the user to exercise with high exercise intensity by allowing the vibration unit 32 to be differently driven from the cycle of vibration in the case where the cycle of vibration being generated exceeds the above-described upper limit.

### Case of Running (Normal Running)

On the other hand, in the case where the kind of exercise determined in Step S24 is normal running, the informing control unit 78 allows the informing unit to inform the user of the load state at the time or during a period when the informing conditions set by the condition setting unit 74 are satisfied. For example, as illustrated in FIG. 11, the informing control unit 78 allows the informing unit 3 to inform the user at a time point P4 when the pulse rate exceeds a lower limit D1 (the same value as the upper limit A2 of the fat burning zone) of the aerobic exercise zone. Further, the informing control unit 78 allows the informing unit 3 to inform the user and the informing unit 3 informs the user of exercise with exercise intensity not reaching the aerobic exercise zone being performed during a period t4 when the pulse rate is lower than the lower limit of the aerobic exercise zone.

### Case of Running (High-Load Running)

FIG. 12 is a diagram illustrating an informing example in the case where the kind of exercise is changed into high-load running from normal running. Further, a straight line D1 in FIG. 12 indicates the lower limit of the aerobic exercise zone and a straight line D2 indicates the upper limit of the aerobic exercise zone.

In the case where the kind of exercise determined in Step S24 is normal running and then the kind of exercise determined in Step S28 is high-load running (last spurt), informing control unit 78 sets the informing conditions again when the kind of exercise is changed. That is, in FIG. 12, the informing condition in normal running is set from the start of measurement to the time point of a time E1, and the informing condition in high-load running is set after the time point of the time E1. In such a case, the informing control unit 78 informs the user of the load state at the time or during a period when the informing conditions set by the condition setting unit 74 are satisfied.

For example, as illustrated in FIG. 12, the informing control unit 78 allows the informing unit 3 to inform the user at a time point P5 when the pulse rate exceeds the lower limit D1 of the aerobic exercise zone and the informing unit 3 informs the user of exercise (normal running) belonging to the aerobic exercise zone being performed. Further, in a case where the heartbeat (pulse rate) are drastically increased in a predetermined time (for example, 10 seconds) and the moving velocity of the user is increased, it is determined that the kind of exercise is high-load running and the informing control unit 78 allows the informing unit 3 to perform the informing process based on the informing conditions in high-load running. That is, after the pulse rate is in the aerobic exercise zone, informing is performed based on the informing conditions of normal running from a time point P6 to a time point of a time E1 and informing is performed based on the informing conditions of high-load running after the time E1 during a period t5 for which the pulse rate exceeds the upper limit D2 of the aerobic exercise zone. In this manner, the informing control unit 78 allows the informing unit 3 to inform the user and the informing unit 3 informs the user of exercise with extreme exercise intensity being performed during a period t5 for which the pulse rate exceeds the upper limit D2 of the aerobic exercise zone.

In addition, since the informing process based on the informing conditions of high-load running is performed after the time E1, informing of the exercise intensity is not performed in the case where the pulse rate is lower than the aerobic exercise zone.

### Case of Running (Interval Training)

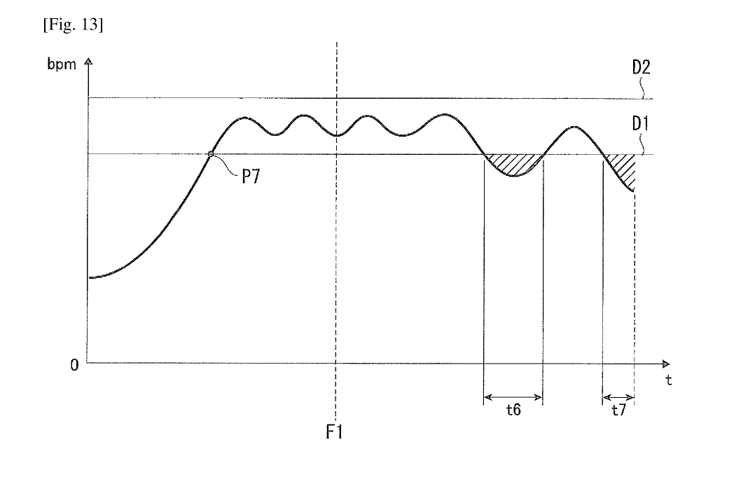

FIG. 13 is a diagram illustrating an informing example in the case where the kind of exercise is changed from normal running into interval training. Further, a straight line D1 in FIG. 13 indicates the lower limit of the aerobic exercise zone and a straight line D2 indicates the upper limit of the aerobic exercise zone.

On the other hand, in the case where the kind of exercise determined in Step S24 is normal running and then the kind of exercise determined in Step S28 is interval training, the informing control unit 78 sets the informing conditions again while the kind of exercise is changed. That is, as illustrated in FIG. 13, the informing condition in normal running is set from the start of measurement to the time point of a time F1, and the informing condition in interval training is set after the time point of the time F1.

For example, the informing control unit allows the informing unit 3 to inform the user at a time point P7 when the pulse rate exceeds the lower limit D1 of the aerobic exercise zone and the informing unit 3 informs the user of exercise (normal running) belonging to the aerobic exercise zone being performed. Further, when high-load exercise and low-load exercise are repeated in the aerobic exercise zone and the exercise intensity fluctuates predetermined times (for example, two times), it is determined that the kind of exercise is interval training and the informing unit 3 performs the informing process based on the informing conditions in interval training. That is, after the pulse rate is in the aerobic exercise zone, informing is performed based on the informing conditions of normal running from a time point P7 to a time point of a time F1 and informing is performed based on the informing conditions of high-load running after the time point of the time F1.

For example, after the pulse rate is in the aerobic exercise zone, the informing control unit 78 allows the informing unit 3 to inform the user and the informing unit 3 informs the user of the exercise with exercise intensity not reaching the aerobic exercise zone being performed during periods t6 and t7 for which the pulse rate is lower than the lower limit D1 of the aerobic exercise zone.

### Effects of Third Embodiment

According to the biological information measuring device 1A of the third embodiment described above, there are following effects.

In the biological information measuring device 1A, the informing conditions according to the kind of exercise of the user analyzed based on the biological information and the motion information detected by the exercise detecting unit 4A are set by the condition setting unit 74, and the informing unit 3 informs the user of the load state (user state in a broad sense) in the case where it is determined that the informing conditions are satisfied by the condition determining unit 75. That is, the informing conditions can be changed according to the kind of current exercise of the user and informing can be performed based on the informing conditions and the current exercise state of the user. Therefore, the load state of the user can be effectively informed and convenience of the biological information measuring device 1A can be improved.

In the case where the kind of exercise of the user is walking, the exercise intensity is informed when the biological information exceeds the upper limit A2 of the fat burning zone to be set according to walking or when the biological information is lower than the lower limit A1 thereof. Accordingly, for example, in the case where the detected biological information (pulse rate) exceeds the upper limit A2 thereof or the detected biological information is lower than the lower limit A1 thereof, the load state (exercise intensity) is informed, and thus, it is possible to encourage the user to correct the load state (exercise intensity) of walking. That is, it is possible to make the user recognize that the current exercise of the user is not an exercise with high fat burning efficiency.

Meanwhile, in the case where the kind of exercise of the user is running, the load state is informed when the biological information exceeds the upper limit D2 of the aerobic exercise zone to be set according to running or when the biological information is lower than the lower limit thereof. Accordingly, in the case where the detected biological information (pulse rate) exceeds the upper limit D2 of the aerobic exercise zone or the detected biological information is lower than the lower limit D1 (A2) thereof, the load state is informed, and thus, it is possible to encourage the user to correct the load state (exercise intensity) of running. That is, it is possible to make the user recognize that the current exercise of the user is not an aerobic exercise.

Therefore, the load state in exercise of the user can be effectively informed.

In the case where the kind of exercise is high-load running (last spurt), when the detected biological information exceeds the upper limit D2 of the aerobic exercise zone to be set according to high-load running, the load state of the user is informed. In this manner, it is possible to encourage the user to correct the load state (exercise intensity) because of continuation of high-load running. That is, it is possible to make the user recognize that the current load state of the user is not appropriate.

In the case where the kind of exercise is interval training, when the detected biological information is lower than the lower limit D1 of the aerobic exercise zone to be set according to interval training, the load state of the user is informed. In this manner, it is possible to encourage the user to correct the load state (exercise intensity) of interval training. That is, it is possible to make the user recognize that the current load state of the user is not appropriate.

In the case where the kinds of exercise are normal running, high-load running, and interval training, since at least one of the upper limit D2 and the lower limit D1 of the aerobic exercise zone is set to be higher than the upper limit A2 and the lower limit D1 of the fat burning zone when the kind of exercise is walking, it is possible to prevent informing that may cause misunderstanding of the load state of the user.

The kind of exercise of the user is analyzed based on the positional information in addition to the biological information (pulse rate) and the motion information (acceleration data) of the user. Consequently, the moving velocity of the user can be obtained by obtaining a change of the positional information. That is, according to the present embodiment, since the kind of exercise can be performed based on the positional information in addition to the biological information and the motion information, it is possible to more reliably perform the analysis of the kind of exercise.

The range of the biological information for each kind of exercise varies for each user because the range thereof is set based on the maximum pulse rate of the user. Consequently, it is possible to set the range of the biological information suitable for both of a user unaccustomed to the exercise and a user accustomed to the exercise such as an athlete.

### Modification of Embodiment

The invention is not limited to the above-described respective embodiments and includes modification or improvement within the range capable of achieving the purpose thereof.

In the above-described respective embodiments, the biological information to be detected by the biological information detecting unit 41 may not be the pulse wave or the pulse rate. For example, biological information may be other kinds of information such as brain wave and heartbeat.

Further, the measuring devices 1 and 1A are configured as wristwatch type devices and the wearing sites of the measuring devices 1 and 1A are set to the wrist of the user, but the invention is not limited thereto. For example, the devices may be worn on the ankle, chest, and abdomen of the user.

According to the respective embodiments, the informing unit 3 may include a voice output unit. In this manner, the load state of the user can be output by the voice.

In the above-described first and second embodiments, the kind of behavior to be analyzed by the behavior analysis unit 73 is not limited to the above-described kinds. For example, the behavior of the user to be classified into exercise may be more specifically analyzed into the kinds of exercise such as bicycle running or climbing.

In addition, according to the respective embodiments, the informing control unit 78 of the measuring devices 1 and 1A allows the vibration unit 32 to vibrate while the informing control unit allows the informing unit 3 to inform the user of the load state. However, the invention is not limited thereto. For example, flickering or lighting of the LED of the display unit 31 is possible in place of the vibration of the vibration unit 32. Further, flickering or lighting of the LED of the display unit 31 in accordance with the vibration of the vibration unit 32 is possible.

According to the respective embodiments, the triaxial acceleration sensor is used as the acceleration sensor which is included in the motion information detecting unit 42. However, the invention is not limited thereto. For example, the acceleration sensor may be at least a biaxial acceleration sensor.

For example, the motion information detecting unit 42 may include an acceleration sensor having two axes substantially orthogonal to each other or an acceleration sensor having four or more axes crossing to one another in three-dimension. Moreover, the motion information detecting unit 42 may have a configuration including another sensor such as a gyro sensor or a pressure sensor in place of or in addition to the acceleration sensor. In addition, the acceleration value detected by the motion information detecting unit 42 may be used for a process of reducing noise caused by the body motion superimposed on a pulse wave signal detected by the biological information detecting unit 41 which is a pulse wave detecting unit.

According to the first and second embodiments, detection of the biological information and detection of acceleration data in Steps S11 and S12 are not limited to the above-described procedures. For example, detection of the biological information and detection of the acceleration data may be started at the same time and detecting procedures may be reversed. That is, as long as the detection of the biological information and the detection of the acceleration data are continuously performed and are stored in the storage unit 6, processes of Steps S11 and S12 may be performed in any order.

Further, according to the third embodiment, the description is made that the processes of Steps 21 to 23 are performed in order of detection of the biological information, detection of the acceleration data, and detection of the positional information. However, the invention is not limited thereto. For example, detection of the biological information, detection of the acceleration data, and detection of the positional information may be started at the same time and detecting procedures may be reversed. That is, as long as detection of the biological information, detection of the acceleration data, and detection of the positional information are continuously performed and are stored in the storage unit 6, processes of Steps S21 to S23 may be performed in any order.

According to the first and second embodiments, informing of the load state is regulated by the informing regulation unit 77 in the case where the kind of behavior is sleep. However, the invention is not limited thereto. When the pulse rate is abnormally increased, for example, the pulse rate exceeding the upper limit A2 (C2) of the fat burning zone (stress zone) is detected even in the case where the kind of behavior is sleep, the informing unit 3 may inform the user.

According to the first and second embodiments, in the case where the kind of behavior is non-exercise, informing using the informing unit 3 may be regulated by the informing regulation unit 77 in the same manner as the case where the kind of behavior is sleep. According to this, since the informing unit 3 does not inform the user while the user has a psychological load, it is possible to prevent the user from further having the psychological load due to the informing.

According to the first embodiment, the lower limit A1 of the fat burning zone is the same as the threshold value B1 thereof. However, the invention is not limited thereto. For example, the threshold value B1 may be set to be higher or lower than the lower limit A1. In this case, the condition determining unit 75 may determine whether the lower limit exceeds the threshold value or a threshold value determining unit that determines whether the lower limit exceeds the threshold value may be separately provided. That is, in the case where the kind of behavior is exercise and the case where the kind of behavior is non-exercise, the threshold value (threshold value included in the informing conditions) used for determining whether to perform informing using the informing unit 3 can be appropriately changed.

According to the second embodiment, in the case where the kind of behavior is non-exercise, the condition setting unit 74 changes the fat burning zone into the stress zone, but the zone may not be changed.

According to the third embodiment, the load state is informed when the biological information is lower than the lower limit of the aerobic exercise zone in the case where the kind of exercise is interval training and the load state is informed when the biological information exceeds the upper limit of the aerobic exercise zone in the case where the kind of exercise is high-load running. However, the invention is not limited thereto. For example, even in the case where the kind of exercise is any one of interval training or high-load running, the informing control unit 78 may allow the informing unit 3 to inform the user when the biological information exceeds the upper limit of the aerobic exercise zone or is lower than the lower limit thereof in the same manner as the case where the kind of exercise is normal running.

According to the third embodiment, the exercise detecting unit 4A includes the positional information detecting unit 43. However, the invention is not limited thereto. For example, the positional information detecting unit 43 may not be provided. In this case, the exercise analysis unit 79 may analyze the kind of exercise based on the biological information (pulse rate) and the motion information (acceleration data) detected by the biological information detecting unit 41 and the motion information detecting unit 42.

According to the respective embodiments, the condition setting unit 74 sets the zones (the fat burning zone and the aerobic exercise zone) of the biological information according to the user. However, the invention is not limited thereto. For example, the ranges (the fat burning zone and the aerobic exercise zone) of the biological information may be obtained as the data through communication using an external device and the communication unit 5.

In addition, the ranges of the biological information may be uniformly determined without being set for each user. That is, it is preferable that the different ranges of the biological information to be set for the kind of exercise be changed in accordance with the kind of detected exercise of the user.

According to the first and second embodiments, the informing control unit 78 allows the vibration unit 32 to be driven such that the cycle of vibration to be generated becomes relatively short. However, the invention is not limited thereto. That is, the informing control unit 78 may not change the cycle of the vibration to be generated.

Further, according to the third embodiment, the informing control unit 78 changes the cycle of vibration to be generated due to the driving of the vibration unit 32 based on the kind of exercise, the informing conditions, and the results in which the informing conditions are satisfied. However, the invention is not limited thereto. That is, the informing control unit 78 may not change the cycle of vibration to be generated.

1.-14. (canceled)

15. A biological information measuring device comprising:
a biological information detecting unit that detects biological information of a user;
a motion information detecting unit that detects motion information related to the motion of the user;
an informing unit that informs the user of a load state of the user;
a behavior analysis unit that analyzes the kind of behavior of the user based on the detected biological information and motion information;
a condition setting unit that sets informing condition of the load state for the behavior;
a condition determining unit that determines whether the informing condition is satisfied according to the kind of behavior; and
an informing control unit that informs the informing unit of the load state when it is determined that the informing condition is satisfied by the condition determining unit.

16. The biological information determining device according to claim 15, wherein the condition setting unit sets the detected biological information to exceed a lower limit of a range of the biological information to be set according to the user as the informing condition in a case where the kind of behavior is at least one of exercise and non-exercise.

17. The biological information measuring device according to claim 15, wherein the condition setting unit sets the detected biological information to exceed an upper limit of the range of the biological information to be set according to the user as the informing condition in the case where the kind of behavior is at least one of exercise and non-exercise.

18. The biological information measuring device according to claim 16, wherein regulation is made such that the condition setting unit sets the detected biological information to be lower than the lower limit of the range of the biological information to be set according to the user as the informing condition in the case where the kind of behavior is exercise and sets the detected biological information to be lower than the lower limit of the range of the biological information to be set according to the user as the informing condition in the case where the kind of behavior is non-exercise.

19. The biological information measuring device according to claim 16, wherein the condition setting unit includes the upper limit of the range of the biological information which is lower than that of the case where the kind of behavior is exercise in the informing condition in the case where the kind of behavior is non-exercise.

20. The biological information measuring device according to claim 15, further comprising an informing regulation unit that regulates informing using the informing unit in a case where the kind of analyzed behavior of the user is sleep.

21. A method of controlling a biological information measuring device that measures biological information of a user and informs the user of a load state of the user, comprising:
detecting biological information of the user;
detecting motion information related to motion of the user;
analyzing the kind of behavior of the user based on the detected biological information and motion information;
determining whether informing condition set for the kind of behavior is satisfied based on the detected biological information and motion information; and
informing the user of the load state of the user when it is determined that the informing condition is satisfied.

22. A biological information measuring device comprising:
a biological information detecting unit that detects biological information of a user;
a motion information detecting unit that detects motion information related to the motion of the user;
an informing unit that informs the user of a load state of the user;
an exercise analysis unit that analyzes the kind of exercise of the user based on the detected biological information and motion information;
a condition setting unit that sets informing condition of the load state for the kind of exercise;
a condition determining unit that determines whether the informing condition is satisfied according to the kind of exercise of the user which is analyzed by the exercise analysis unit based on the detected biological information and motion information; and
an informing control unit that informs the informing unit of the load state when the informing condition is satisfied by the condition determining unit.

23. The biological information measuring device according to claim 22,
wherein the kind of exercise includes at least walking and running, and
the condition setting unit sets at least one of conditions in which the detected biological information exceeds the upper limit of the range of biological information predetermined for the kind of exercise and in which the detected biological information is lower than the lower limit of the range of the biological information as the informing condition in a case where the kind of exercise is one of walking and running.

24. The biological information measuring device according to claim 23,
wherein the running includes high-load running and interval training, and
the condition setting unit sets the detected biological information exceeding the upper limit of the range of the biological information according to the high-load running as the informing condition in a case where the kind of exercise is the high-load running and sets the detected biological information being lower than the lower limit of the range of the biological information according to the interval training as the informing condition in a case where the kind of exercise is the interval training.

25. The biological information measuring device according to claim 24,
wherein the running includes normal running other than the high-load running and the interval training, and
at least one of threshold values of the upper limit and the lower limit of the range of the biological information according to each of the normal running, the high-load running, and the interval training is higher than the threshold value in the range of the biological information according to the walking.

26. The biological information measuring device according to claim 22, further comprising:
a positional information detecting unit that detects positional information of the user, and
wherein the exercise analysis unit analyzes the kind of exercise of the user based on the biological information, the motion information, and the positional information.

27. The biological information measuring device according to claim 22, wherein the range of the biological information for the kind of exercise is set according to the user.

28. A method of controlling a biological information measuring device that measures biological information of a user and informs the user of a load state of the user, comprising:
detecting biological information of the user;
detecting motion information related to motion of the user;
analyzing the kind of exercise of the user based on the detected biological information and motion information;
determining whether informing condition set for the kind of exercise is satisfied based on the detected biological information and motion information; and
informing the user of the load state of the user when it is determined that the informing condition is satisfied.
